(19) United States
(12) Patent Application Publication
(10) Pub. No.: US 2003/0163031 A1
(43) Pub. Date: Aug. 28, 2003

(54) Method and system for providing centralized anatomic pathology services

(75) Inventors: Charles T. Madden, Newport Beach, CA (US); Michael G. Dolan, Orange, CA (US); Glenn R. Ray, Laguna Niguel, CA (US); Michael H. Voss, San Juan Capistrano, CA (US)

Correspondence Address: PENNIE AND EDMONDS, 1155 AVENUE OF THE AMERICAS, NEW YORK, NY, 100362711

(73) Assignee: ADLabs, Inc.

(21) Appl. No.: 10/392,194

(22) Filed: Mar. 19, 2003

Related U.S. Application Data
(63) Continuation of application No. 09/604,290, filed on Jun. 26, 2000, now abandoned.

(51) Int. Cl. A61B 5/00

(52) U.S. Cl. 600/300

(57) ABSTRACT

A method for providing centralized anatomic pathology services. A plurality of regional pathology laboratories is provided, each laboratory servicing a defined geographic region. A master storage (e.g., database) of pathology information is maintained, the storage being accessible by pathologists associated with any regional laboratory via a communications link. Tissue samples requiring pathology processing are collected from a medical entity located in a first geographic region by a regional pathology laboratory. The tissue is processed, and a tissue slide is created. A digital, diagnostic quality image of the tissue slide is created and stored in the master storage. A pathologist, who may be remotely located with respect to the first geographic region, is provided with access to the stored diagnostic image via the communications link, to enable diagnosis by the pathologist without physical possession of the slide. The digital, diagnostic quality image may be compressed before it is stored in the master storage. The diagnosis may be a primary or a secondary diagnosis. In the case of a secondary or supplemental diagnosis, the pathologist is provided with access to any prior analysis and annotations, stored in the master storage, relating to the diagnostic image. A system for implementing the method is also disclosed.

### FIELD OF THE INVENTION

The present invention relates to the field of diagnostic health care and pathology services. More particularly, the present invention relates to a method and system for providing centralized anatomic pathology services through a plurality of regional laboratories and pathology professionals having access to a master storage of diagnostic information.

### BACKGROUND OF THE INVENTION

Anatomic pathology (hereinafter “AP”) laboratories analyze bodily tissues and cells obtained through invasive procedures to identify the nature and treatment of a disease. Within AP labs, there are two distinct sections: technical processing and professional analysis. The technical processing currently performed in most AP labs and hospitals involves a series of procedures starting with a gross tissue sample and concluding with a thin, stained tissue mounted on a slide. The slide is then analyzed by a pathologist under a microscope (i.e., professional analysis). There are also generally two divisions of anatomic pathology services: general and esoteric. General AP looks for disease. Esoteric AP involves further testing to identify the specific disease and method of treatment at the cellular level. Tissue requiring esoteric AP processing is usually referred to a specialty esoteric AP lab.

Hospitals once controlled the market for AP services but several trends have fragmented the market, to the ultimate detriment of both the patient and the AP industry. These trends include ambulatory care, the development of specialty clinics and managed care groups, and the increasing demands for and costs of cancer diagnostic testing, diagnostic technology, and specialized personnel. This fragmentation has reduced the volume of AP testing performed in hospitals, increased the cost of such testing, and spread the number of qualified AP professionals over a number of small labs. To date, attempts to counteract this fragmentation and centralize anatomic pathology services have involved the purchasing of individual pathology practices and the consolidation of those practices into a regional lab. Funding for AP services has also been significantly reduced by the Balanced Budget Act (BBA) and Ambulatory Pricing Codes (APC). The cost pressures, reduced volume, and reduced pricing have impacted quality and fueled the trend to outsource all AP lab services.

AP labs, unlike clinical labs that are highly automated and employ standardized tests to analyze bodily fluids, are neither automated nor standardized. This lack of standardization, together with the number of steps required to process tissue samples, has resulted in an unacceptably high diagnostic error rate documented at 5% (and probably in reality much higher). See Second Opinions: Researchers Say Second Review Needed, California Healthline, Dec. 2, 1999. This is an extremely high diagnostic error rate for services so intimately linked with potentially life-threatening diseases.

This high error rate is likely due to a combination of factors caused by fragmentation, a lack of standardization, the multiple steps in tissue processing and an extreme reliance on the individual capabilities of multiple technicians and pathologists in the AP industry. Another contributing factor is the outdated technology available to the pathologist, who must observe the tissue sample through the narrow depth of field of a microscope, a 400-year-old technology limited to selected bands of visible light.

Ultimately, the attending physician and his or her patient are highly dependent on this chain of AP processes. The attending physician must often consult with the pathologist on appropriate treatment protocols to treat an identified carcinoma. If a disease is missed, or mis-diagnosed, the results for the patient can be tragic in both a life cut short, or an unnecessarily burdensome treatment protocol.

Adding to the problems discussed above is the high level of fragmentation in a number of post-diagnostic areas. Since most AP labs are not equipped to provide esoteric services, the AP labs must pack and ship the original tissue blocks to esoteric AP labs for additional testing. Similarly, if a secondary diagnosis is required, the AP lab must pack and ship its processed tissue slides to another lab or pathologist for a peer review or second opinion. The additional time required to package and ship either tissue blocks for esoteric testing or slides for an expert review or specialty second opinion is particularly inefficient. Further, when a secondary diagnosis is desired and the slides are shipped to a secondary pathologist, the primary and secondary pathologists are not able to simultaneously view and discuss the slides in the case of disagreement.

In addition, patient information, including diagnostic and treatment histories, protocols, or resulting population epidemiologies, that could otherwise be used to enhance the capabilities and training of medical professionals and continually improve performance, is often scattered among multiple institutions, physicians offices, and ancillary facilities. As a result, information regarding a cancer diagnosis, prognosis, treatment plans and results must often be gathered from multiple sources.

In summary, the present state of AP services is characterized by little standardization or automation, few assisting technologies, and a resulting diagnostic product (e.g., tissue slides) that inhibits concurrent second opinions and peer review. The dependence on a serial chain of professionals and procedures induces the possibility of multiple errors. Despite its strong reliance on unique professional skills, industry fragmentation has caused a broad distribution of pathology professionals of varying capability and quality throughout the country. This fragmentation further diminishes the ability of pathologists to practice in their particular areas of sub-specialization. It would therefore be desirable to provide a method and system to ameliorate the problems facing the AP industry and reduce the diagnostic error rate associated with AP services.

### SUMMARY OF THE INVENTION

In a preferred embodiment, the present invention is directed to a method and system for providing centralized anatomic pathology services. A plurality of regional pathology laboratories are provided, each laboratory servicing a defined geographic region. A master storage (e.g., database) of pathology information is maintained, the storage being accessible by pathologists associated with any regional laboratory via communications links. Tissue samples requiring pathology processing are collected from a medical entity located in a first geographic region by the region's pathology laboratory. The tissue is processed, and a tissue slide is created. A digital, diagnostic quality image of the tissue slide is created and stored in the master storage. The image may be compressed before storage. A pathologist, who may be remotely located with respect to the first geographic region, is provided with access to the stored diagnostic image via the communications link, enabling the pathologist to render a diagnosis without having physical possession of the tissue slide. Any auditory analysis prepared by the pathologist relating to the diagnostic image may be stored in the master storage in linked relation to the stored diagnostic image. In addition, a transcribed, textual version of the pathologist's analysis may also be stored in linked relation to the image. Pathologists viewing the image may add annotations using, for example, a digital pen. These annotations may be stored as a separate overlay file so that the image may be subsequently viewed with or without the annotated overlay as described below. The master storage of pathology information may include diagnostic treatment information (e.g., treatment protocols, etc.). The diagnosis may be a primary diagnosis or a secondary diagnosis (e.g., peer review, specialty diagnosis, etc.). If the diagnosis is a secondary diagnosis, the pathologist may be provided with access to any prior comments or analysis and annotated overlays relating to the diagnostic image. In addition, primary and secondary pathologists are able to view the tissue slides concurrently. Preferably, the regional pathology laboratories of the present invention contract directly with health institutions (e.g., hospitals, clinics, etc.) to perform those institutions' tissue processing without purchasing existing pathology practices.

In another aspect, the present invention relates to a method and system for providing centralized anatomic pathology services comprising: a scanning device for creating a digital, diagnostic quality image of a tissue slide; a server computer including a database configured to store pathology information, including the digital, diagnostic quality image and any analysis and annotations relating to the image; a processor configured to compress the digital, diagnostic quality image prior to storage in the database and to provide access to pathology information, including the image, to an authorized user to enable a remote diagnosis by the user; and communications links connecting a plurality of regional pathology laboratories and other authorized users to the server computer.

### BRIEF DESCRIPTION OF THE DRAWINGS

The present invention will be understood and appreciated more fully from the following detailed description, taken in conjunction with the drawings in which:

FIG. 1 is a block diagram illustrating the flow of anatomic pathology services in the prior art;

FIG. 2 is a block diagram illustrating the structure and operation of a preferred embodiment of the present invention;

FIG. 3 is a flowchart depicting a preferred embodiment of the present invention;

FIG. 4 is block diagram depicting another preferred aspect of the present invention; and

FIG. 5 is a block diagram illustrating digital imaging of tissue slides in accordance with a preferred embodiment of the present invention.

### DETAILED DESCRIPTION OF THE PREFERRED EMBODIMENTS

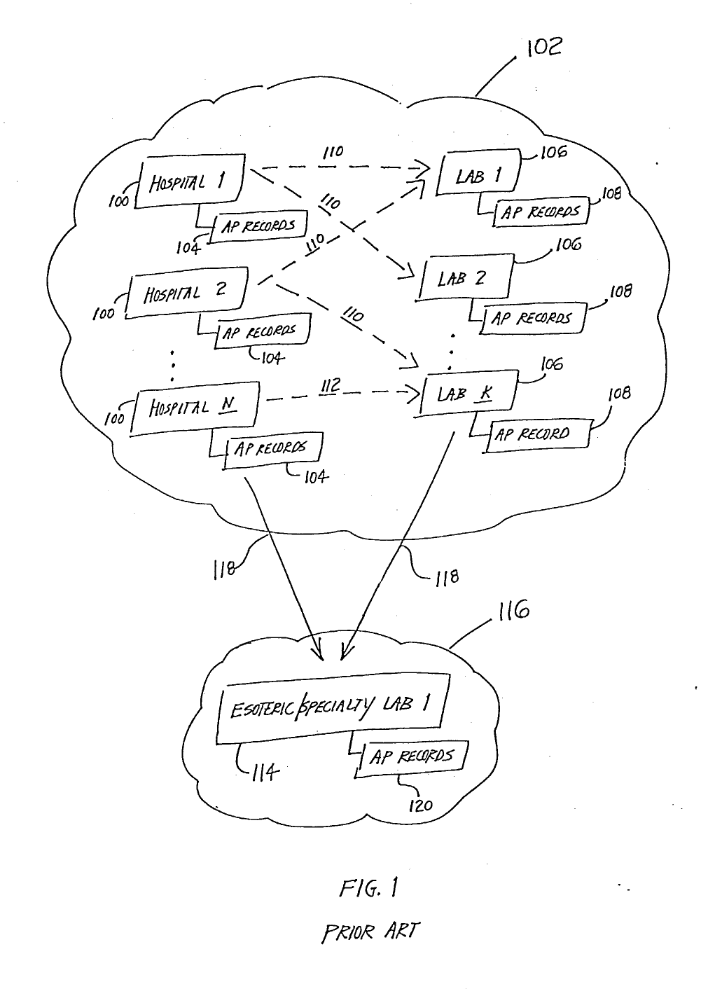

Reference is now made to FIG. 1, which is a block diagram illustrating the flow of anatomic pathology services in the prior art. Hospitals 100 in a given geographic region 102 may perform some anatomic pathology (hereinafter “AP”) services and keep their own set of AP records 104. Hospitals 100 may also outsource some, or all, of their AP processing to AP labs 106 in the same geographic region 102. Each lab 106 will also keep its own set of AP records 108 for a given case. As shown in FIG. 1, a given hospital 100 may outsource its AP work to multiple AP labs 106 (shown by arrow paths 110), or only a single AP lab 106 (shown by arrow path 112). In addition, some tissue samples from both hospitals 100 and AP labs 106 may require analysis by an esoteric lab 114, which may be located in a different geographic region 116. Thus, hospitals 100 and labs 106 must physically package and ship a given tissue slide to esoteric lab 114 (shown by arrow paths 118). As one might expect, the time delay inherent in this configuration prevents concurrent peer review and secondary diagnoses. In addition, esoteric lab 114 will maintain its own set of pathology records 120, separate from the hospital records 104 and general AP lab records 108. As a result, any pathologist or patient who wishes to assemble comprehensive diagnostic information/records in a given case must contact each institution that performed AP services and request such records because each institution maintains its own archive system.

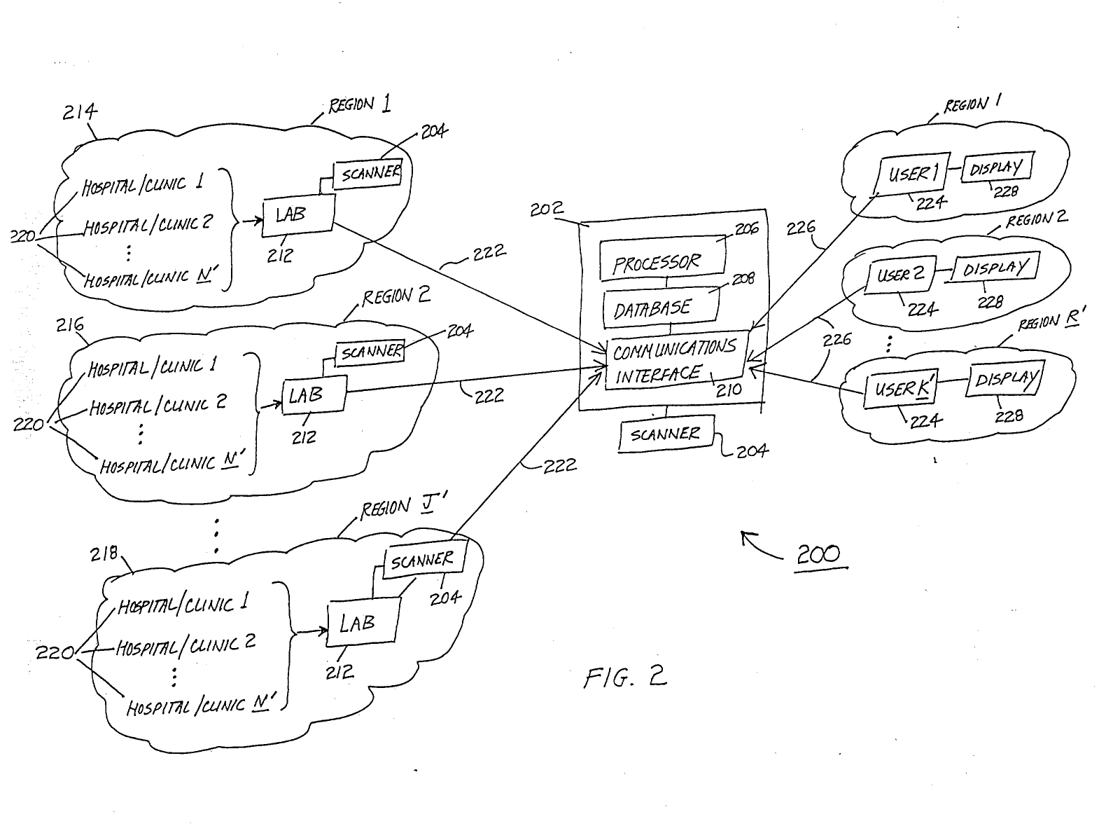

Reference is now made to FIG. 2, which is a block diagram illustrating operation of a preferred embodiment of the present invention. As shown, a system 200 comprises a server computer 202, including a scanning device or other imaging equipment 204, a processor 206, one or more databases 208, and a communications interface 210. A network of regional labs 212 is provided, with each lab servicing a given geographic region (e.g., a metropolitan region like Los Angeles), shown as Region 1 (214), Region 2 (216), . . . , Region J′ (218). As shown, scanners 204 may be located on site at the server computer and/or at each regional lab 212. As used herein, the term scanner refers to a device for creating a digital image of an object, such as a tissue sample. In a preferred embodiment, scanner 204 may comprise a linear Charge Coupled Device (CCD) as described in more detail below. Each laboratory 212 collects tissues requiring pathology processing from one or more hospitals, doctor's offices, clinics, and other medical entities 220 within its assigned geographic region 214, 216, 218. Preferably, each regional laboratory 212 contracts directly with health institutions 220 (e.g., hospitals, clinics, etc.) to perform those institutions' tissue processing without purchasing existing pathology practices. Each laboratory 212 then processes its tissue samples and creates diagnostic quality, digital images of the samples using scanner 204. Creation of these images is discussed in more detail below. Each lab 212 transmits digital images that it has created of collected tissue samples to server 202 via communications links 222 (e.g., wired or wireless pathways) and communications interface 210. These images may be compressed before transmission to server 202 or may be compressed at server 202 by processor 206. Processor 206 receives the images and stores them in database 208. Database 208 is also configured to store other types of pathology information, including accompanying analysis and annotations relating to the digital images, diagnostic treatment protocols, and patient information. Such analysis and annotations includes auditory comments, transcribed textual versions of such comments, and annotated image overlays highlighting areas of interest on the image. Suitable databases for storing such data are available from Oracle Corp. of Redwood City, Calif. It should be understood that server 202 may be physically located at one of regional labs 212, off-site but within the same geographic region as one of regional labs 212, or at some other location remote from regional labs 212.

As noted, in a preferred embodiment, the digital images generated by the present system may be compressed before transmission or storage. Compression is desirable because each diagnostic-quality digital image may be quite large. In particular, a diagnostic quality digital image of a single slide may typically be approximately 500 MB in size. Images of this size would require enormous quantities of memory space. Moreover, the present system contemplates that these diagnostic-quality images may be transmitted to remotely located pathologists for review and diagnosis, as described in more detail below. In many cases, however, the available bandwidth for transmitting images to these pathologists may be 1 Mb/s or less. At that rate, it would take several minutes to transmit a single uncompressed image. Thus, compression is desirable to reduce image transmission time and facilitate the pathological diagnosis system contemplated by this application.

In a preferred embodiment, the compression ratio employed to compress the diagnostic-quality digital images of the present system may be not less than 50:1. In this preferred embodiment, each image may typically require approximately 10 MB of storage and may be transmitted in less than 80 seconds (10 MB/(1 Mb/s). In a further preferred embodiment, the compression ratio may be not less than 100:1. In this preferred embodiment, each image may typically require approximately 5 MB of storage and may be transmitted in less than 40 seconds (5 MB/(1 Mb/s)).

It has been determined, however, that many prior art compression algorithms, such as those employed by TIFF, JPEG, and GIF, are not suitable for the present system when used to compress images of tissue slides at the preferred compression ratios described above. This is because the amount of detail in a tissue slide is not uniform throughout the slide. Rather, some areas of the slide contain significant detail, while others contain much less. At high compression ratios, however, most prior art compression algorithms introduce severe pixelization distortions and artifacts into such images, and thus are not suitable for the pathological images of the present system.

More specifically, many prior art compression algorithms divide an image into discrete blocks and compress each block individually. The compression ratio applied to each block is independent of the block's content and does not take into account the degree of detail in the block. Consequently, blocks containing significant detail are compressed to the same degree as those containing little detail. This results in severe artifacts in high-detail regions of the image, especially as the compression ratio is increased.

It has therefore been determined that a preferred compression algorithm for the present system is one that differentiates between areas of high detail and low detail in a digital image and applies a higher compression ratio to areas of low detail while applying a lower compression ratio to areas of high detail within the same image. In a preferred embodiment, the present system may employ a wavelet compression algorithm to compress each diagnostic-quality digital image. Wavelet compression has been found desirable for use in the present system because it possesses the desirable quality described above, i.e., it differentiates between areas of high detail and low detail within an image and compresses the areas of high detail less than those of low detail. As a result, it does not introduce diagnostically significant distortions or artifacts that would affect a pathologist's diagnosis of the tissue rendered in the image even at relatively high compression ratios. Specifically, wavelet compression can compress digital images approximately 80 times without introducing artifacts or distortions that would affect a pathologist's ability to diagnose the tissue rendered in the digital image.

The use of wavelet compression also provides added benefits in the context of the present system. For example, wavelet compression facilitates computer-aided analysis of diagnostic images (e.g., object recognition), so that tissue patterns which are symptomatic of particular types of cancers may be recognized by computer analysis. Such computer analysis may be used to supplement, or possibly even replace, diagnosis by a human pathologist. Suitable wavelet compression software is available from Summus, Ltd. of Raleigh, N.C.

Returning to FIG. 2, authorized users 224, such as pathologists affiliated with a regional lab 212, may also access server 202 via communications links 226 (e.g., wired or wireless pathways) and communications interface 210. Users 224 may be located on site at regional labs 212 or at other locations remote from both the regional labs 212 and server 202. For example, a user 224, typically a pathologist, may download a compressed image, or receive such an image via electronic mail, for viewing on a display 228 and subsequent diagnosis. Suitable monitors for viewing such images are available from Sony Electronics Inc. of Park Ridge, N.J.

If there have been prior diagnoses, user 224 may also access the diagnostic reports, as well as analysis and annotations, of fellow pathologists relating to the image. Upon completion of his or her diagnosis, the pathologist can record and store a diagnostic report, including auditory comments, in database 208. The diagnostic report will also include a transcribed version of the pathologist's auditory comments and one or more annotated overlays highlighting and describing areas of interest on the image that may be created by the pathologist while viewing the image by using, for example, a digital stylus or touch-screen and appropriate software tools. Other pathologists may then access this diagnostic report from remote locations to review or supplement the diagnosis. A pathologist rendering a supplemental diagnosis may create additional files with auditory comments and transcribed versions of those comments. The original image file, the original auditory comments file (i.e., voice file), and the original transcribed version of the auditory comments are preferably designated “read-only” files. A pathologist rendering a supplemental diagnosis may, however, create additional annotated overlays for the image that may be displayed alone or in combination with another annotated overlay prepared by the first pathologist. Alternatively, the second pathologist may be given editorial rights to the first pathologist's comments, analysis, and/or annotations. This may be appropriate, for example, if the second pathologist is a supervisor of the first pathologist. Thus, the present system enables multiple simultaneous opinions and concurrent multiple second opinions or peer reviews.

As another example of the functionality of the present system, when user 224 is, for example, an attending physician, he or she can access server 202 and review a given patient's diagnosis. The attending physician can also access diagnostic treatment protocols stored in database 208 related to the particular disease diagnosed to better advise both the patient and his or her family members.

As still another example of the functionality of the present system, a pharmaceutical company may be granted access to database 208 so that it may utilize information stored in the database for cancer research and product development purposes.

While in the above description all inpatient and outpatient institutions, providers, attending physicians, and pathologists are linked in a closed network (i.e., an intranet) due to the private and sensitive nature of the patient information stored on database 208, it is contemplated that the system of the present invention may be implemented over an open network, such as the Internet, with appropriate safeguards, such as encryption of private patient information.

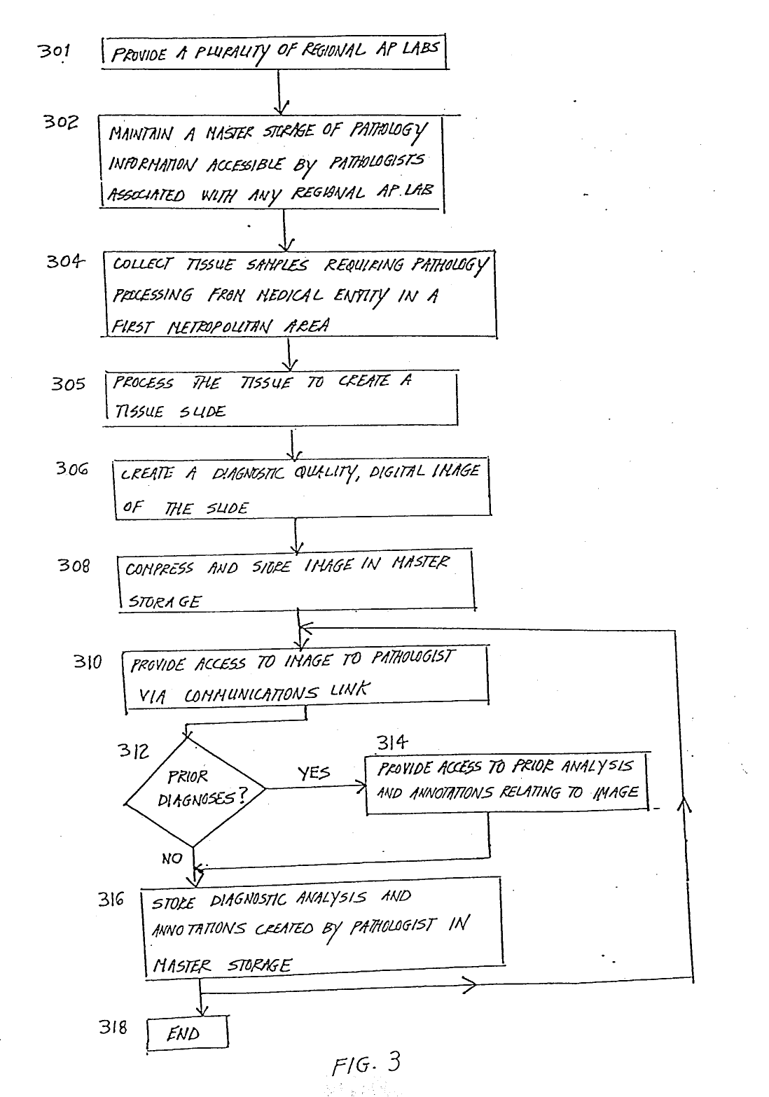

Reference is now made to FIG. 3 which is a flowchart depicting a preferred embodiment of the present invention. In step 301, a plurality of regional AP labs (shown as 212 in FIG. 2) are provided across the country, each lab servicing a defined geographic region 214, 216, 218. All tissue processing in a given region is consolidated in these labs. Each lab may be centrally located within its given region for convenience. The AP labs provide all of the necessary diagnostic procedures in anatomic pathology, cytology, histology, special stains, immunohistochemistry (IHC), flow-cytometry and molecular biology (i.e., both generic and esoteric AP services). Pathologists affiliated with a regional lab may work on site at the lab itself, or from a location remote from the lab, as will be described more fully below. Regional labs service a given geographic region preferably defined by a radius of up to 100 miles for general AP services and up to 1500 miles for esoteric AP services. Smaller regions defined by radii of approximately 50-60 miles may also be employed.

In step 302, a master storage (i.e., one or more databases 208) of pathology information is maintained, the storage being accessible by pathologists and other authorized users affiliated with a regional lab 212. All inpatient and outpatient institutions, providers, attending physicians, and pathologists are linked to a single web-based intranet server and data system (i.e., a closed network). All diagnostic and treatment protocols, along with other medical information, are consolidated in this storage. In step 304, tissue samples requiring pathology processing are collected from one or more medical entities (e.g., hospitals, doctor's offices, clinics, etc.) in a given geographic area. The collecting is preferably performed by an agent of the regional lab (e.g., a courier) located in the same geographic region as the medical entity.

In step 305, a tissue slide for the sample is created. As known in the art, as part of step 305 the tissue sample is typically grossed, processed (i.e., fat and water are removed from the sample), embedded in paraffin, sliced into thin sections, mounted on a labeled slide, and stained. In the prior art, such slides would normally be viewed by a pathologist under a microscope. In contrast, in a preferred embodiment of the present system a diagnostic quality, digital image of the slide is created as depicted in step 306. The creation of such diagnostic quality images is discussed more fully below. The image is then compressed and stored in the master storage in step 308.

In step 310, an affiliated pathologist wishing to view the image and provide a diagnosis is provided with access to the image via a communications link (shown as 226 in FIG. 2). The affiliated pathologist may be located on site at the lab that actually processed and scanned the tissue sample, at another affiliated lab (either general or esoteric) in a different region, or at any other location (e.g., home office) where the pathologist can communicate with the storage and view the image. It is also contemplated that compressed images may be transmitted to a pathologist at a remote location via electronic mail, typically during nighttime or other off-line time. Consequently, when the pathologist decides to view an image, it will be fast, and efficient. If there have been prior diagnoses, the pathologist is also provided with access to any analysis (e.g., dictated comments, textual reports, annotated overlays) relating to the image as depicted in steps 313, 314. The pathologist may then render a diagnosis and create and store a diagnostic report, including auditory comments, in step 316. It is also contemplated that the pathologist's auditory comments will be transcribed into a textual report linked to the image. Steps 310 through 316 are then repeated, if necessary, until the diagnosis is deemed adequate and the process ends in step 318. A graphical user interface (GUI) may be provided to facilitate the pathologist's use of the present system.

Thus, the present invention provides for transmission of diagnostic reports and images to attending physicians and enables concurrent multiple opinions regardless of the physical location of the tissue sample or the diagnosing pathologists.

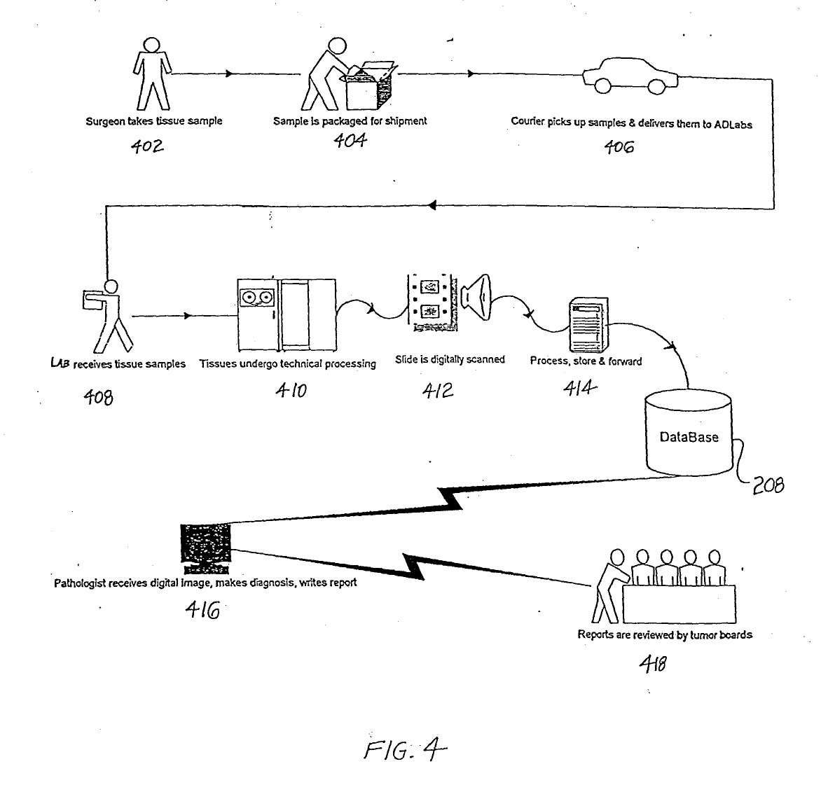

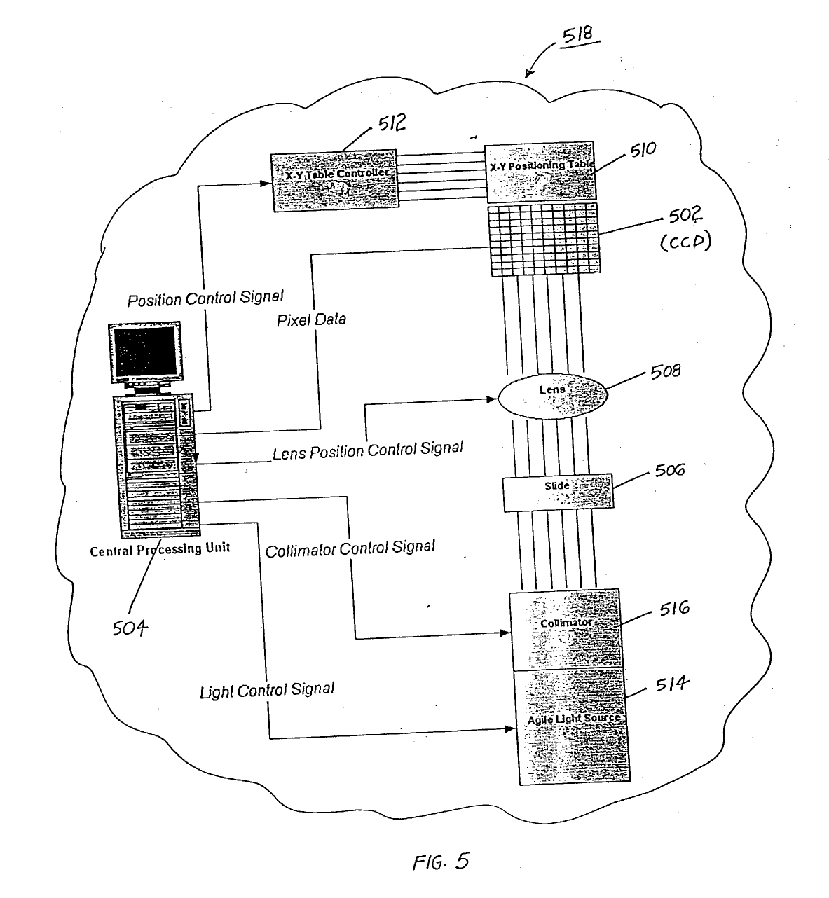

Reference is now made to FIG. 4, which is a combined flowchart/block diagram depicting a preferred embodiment of the present invention, in combination with FIG. 5, which is a block diagram of a preferred apparatus 518 for digitally imaging tissue slides. As shown at 402 in FIG. 4, a surgeon practicing at a hospital, doctor's office, or other medical institution in a given region removes tissue samples from a patient. At 404, those tissue samples are packaged and, at 406, transported to a local AP lab 212 for processing. After AP lab 212 receives the samples, they are accessioned, grossed, processed (e.g., embedded, sliced, stained, and mounted) and a tissue slide is created, as shown at 408 and 410, respectively. As shown at 412, the slide is then scanned to create a digital image of the tissue sample.

With reference to FIG. 5, in a preferred embodiment, a tissue slide 506 may preferably be scanned using scanning apparatus 518 which comprises a charge coupled device (CCD) 502. As known in the art, a CCD is a light sensitive solid-state device composed of thousands or even millions of tiny cells. Any light falling on a cell is converted into a charge, which is then measured by the CCD's electronics and represented by a number (e.g., the number may range from 0 (no light) to 65,535 (extremely intense light)). Collectively, the cells make up a digital image of an object in the field of view of the CCD. A suitable CCD for use in the present system is the Kodak Model KLI8811 Trilinear Color CCD Array available from the Microelectronics Technology Division of the Eastman Kodak Company. It is preferred that the CCD of the present invention be a linear CCD, which will have fewer cells (in the range of 8,000 to 14,000 cells) than, for example, an area CCD. As known in the art, area CCDs of any significant size (e.g., 1K×1K) are very expensive due to significant defect rates in the production of CCD sensors.

Alternatively, other types of digital sensors might be employed, such as CMOS. Although current CMOS models have limited performance capabilities and could not be implemented in the present invention, it is anticipated that acceptable devices will soon be available.

CCD 502 is mounted on a biaxial positioning device 510, such as the Velmex 1500 X-Y Positioning Table available from Velmex, Inc. of Bloomfield, N.Y. Biaxial positioning device 510 is preferably connected to an X-Y table controller 512, such as the Velmex NF90. Under control of a computer 504, X-Y table controller 512 allows a user to manipulate the position of CCD 502 with respect to a tissue slide 506 so as to position slide 506 within the CCD's field of view. Since CCD 502 is, in this preferred embodiment, moved relative to slide 506, and since the exact height of the linear sensor. elements may be determined (typically on the order of 7 microns), movement of CCD 502 may be precisely controlled so that each adjacent partial image of the slide exactly abuts its neighboring images and may be combined to form a single image without significant processing.

Alternatively, the CCD 502 may take the form of a scan back inserted into the back of a standard box view camera. A suitable scan back is the Super8K available from BetterLight, Inc. of San Carlos, Calif. In this embodiment, the arrangement of FIG. 5 may be modified so that CCD 502 remains stationary and tissue slide 506 is moved by biaxial positioning device 510 relative to CCD 502.

A light source 514 directs light through a collimator 516 toward tissue slide 506. A suitable light source is the TLB6000 Series available from Diagnostic Instruments Inc. of Sterling Heights, Mich., and a suitable collimator is the XR-Heliflex available from Rodenstock Precision Optics, Inc. of Rockford, Ill.

Light passing through slide 506 is focused on CCD 502 by a lens 508, such as the Rodagon-G 50 mm f2.8 lens available from Rodenstock Precision Optics, Inc. of Rockford, Ill. Positioning of lens 508 may be controlled by a lens control signal output by computer 504.

Computer 504 is preferably adapted to reconstruct images in the field of view of CCD 502 and to display them on an appropriate computer display, such as a CRT monitor. Computer 504 may be a typical PC workstation such as the Kayak Series workstation available from the Hewlett Packard Company of Palo Alto, Calif.

In a preferred embodiment, the present system employs digital magnification either in combination with, or instead of, optical magnification to magnify tissue samples for pathological examination. This provides significant benefits over microscope-based pathological methods and systems for the following reasons.

As known in the art, the human eye can effectively resolve only five to ten line pairs per millimeter, depending on the individual. Consequently, pathologists are unable to analyze tissue samples with the naked eye, and instead require the aid of some sort of magnification. In the past, such magnification has typically been provided by optical microscopes that magnify tissue samples approximately 400×.

Optical microscopes, however, introduce specific limitations to pathological analysis. First, they are limited to the visible light spectrum. Second, they provide a relatively narrow field of view. Because of this narrow field of view, a pathologist is unable to examine an entire slide at one time, but must instead move the slide under the microscope to see different portions of the tissue sample. Third, the use of optical magnification decreases depth of field. Thus, at high powers of magnification it may be impossible to obtain acceptable focus at all significant depths of the sample, at any one moment.

The present system alleviates these problems by replacing the microscope as the main diagnostic tool. Accordingly, in a preferred embodiment, the present system employs digital magnification either in combination with, or instead of, optical magnification to magnify tissue samples for pathological examination. In a preferred embodiment, the system employs 40× optical magnification during scanning by CCD 502, and then digitally magnifies the scanned image another 10× before display to a pathologist. Thus, the system preferably magnifies tissue samples approximately 400× (i.e., approximately the degree of magnification provided by optical microscopes of the prior art).

The preferred scanning and magnification arrangement of the present system, however, provides significant benefits over purely optical magnification. First, because digital magnification does not affect the depth of field, the depth of field of an image magnified optically 40× and digitally 10× is significantly greater than the same image magnified optically 400×. Second, CCD 502 typically has a field of view as large as 72 mm by 96 mm, and thus is typically able to capture an entire slide in a single image at 3× optical magnification. Alternatively, if the field of view of CCD 502 is too small to capture the entire slide in a single pass, multiple images may be taken of different sections of the slide and then combined as a whole by computer 504. This image may then be digitally magnified for display to the pathologist who is able to view the entire slide at one time.

Moreover, the digital magnification of the present system will not introduce visible artifacts to the displayed image. Specifically, as noted above, the human eye can effectively resolve only five to ten line pairs per millimeter. CCD 502, however, has significantly higher resolution; typically in excess of 55 line pairs per millimeter. Therefore, the scanned image may be digitally magnified as much as approximately 10× without introducing artifacts that would be noticeable to a pathologist.

In a preferred embodiment, lens 508 is capable of providing a resolution significantly higher than that provided by typical microscope lenses. As a result, the digital enlargement provided by the present system is not “empty enlargement”, i.e., magnification that increases the size of the image without increasing its resolution, but rather improves image detail. More specifically, since the human eye cannot resolve more than 5 to 10 line pairs per millimeter, a pathologist cannot resolve all of the detail in an image with a resolution of 55 line pairs per millimeter. When that 55 line pair per millimeter image is digitally enlarged, say 10× times, additional detail that could not previously be resolved by the human eye will be resolvable. Thus, the digital magnification contemplated by the present invention provides an image having a greater level of detail to the diagnosing pathologist.

In contrast, use of conventional microscope optics to magnify a slide would provide only optical magnification of the imaged slides with no possibility of any real (non-empty) further digital magnification. Any further digital magnification of the image results in only “empty magnification,” i.e., magnification that increases the size of the image without increasing its resolution.

More specifically, microscope optics do not generally provide resolution beyond the capacity of the human eye. When digitally enlarged, images at this resolution simply increase in size (because gaps or voids between adjacent pixels appear) but do not provide additional detail. Thus, digital magnification of these images is “empty” magnification rather than real magnification.

Furthermore, as noted above, the human eye, even when aided by an optical microscope is only able to detect visible light, a small band of the electromagnetic spectrum. By contrast, in a preferred embodiment, CCD 502 may be adapted to selectively detect light from specific portions of the electromagnetic spectrum including those outside the visible spectrum (e.g., it may be adapted to detect X-ray, ultraviolet, or infrared light). As described below, this permits pathological study of tissue slides without requiring staining as in the prior art.

More specifically, as known in the art, when a pathologist examines two unstained tissue samples under a microscope, it is difficult to detect differences in tissue density between the samples because the unstained samples absorb visible light in approximately the same way. Consequently, the practice in the prior art has been to stain such samples. Less dense tissue absorbs more stain and hence exhibits a darker color than denser tissue. A pathologist is thus able to discriminate the relative density difference of a stained tissue sample by examining the intensity of its color.

By contrast, in a preferred embodiment of the present system, unstained tissue samples are exposed to non-visible light that is absorbed differently by samples of different density. This difference is preferably detected by CCD 502 which may be provided with suitable filters to permit detection of particular portions of the electromagnetic spectrum. As known in the art, a substitute light source 514 may be furnished to provide either infrared or ultraviolet light. For example, typical halogen bulbs are a good source of infrared light, and the filters associated with such bulbs may be modified to yield infrared light only. Similarly, typical flourescent bulbs are a good source of ultraviolet light and may be modified to yield ultraviolet light only. Displayed images of the scanned samples may be colored in various shades to represent different tissue densities detected by CCD 502.

Thus, this preferred embodiment of the present system provides a way to avoid the staining step required in the prior art. This reduces the time and cost necessary to prepare tissue samples and also decreases the number of steps during which the slide may be improperly processed.

As noted above, images created in the present system have a greater depth of field than those viewed through microscopes that employ, for example, 400× optical magnification. Nevertheless, the depth of field of a single image created by row CCD sensor 502 may not be adequate to ensure that the entire slide is in focus at every depth that may be of interest to a pathologist. Consequently, in a preferred embodiment, multiple images of a tissue sample may be created each of which is in focus for a particular depth of field range. Collectively, these ranges may encompass the entire depth of field that is of interest.

These multiple images may be created in several ways. For example, a multiple-row CCD sensor may be manufactured or oriented so that each sensor row is a different distance from lens 504 during imaging and is therefore focused at a different depth. Output from each row may then be used to create an image focused at a particular depth. Alternatively, a single-row CCD sensor may be used to create the multiple images by scanning the sample several times and changing the relative position of lens 508 and sensor 502 (or alternatively lens 508 and slide 506) between scannings to change the depth that is in focus during each scan.

The multiple images for a single slide may be presented to the pathologist as a logical “stack.” More specifically, when a pathologist calls up particular slide, he or she may first be shown the image that has in focus the depth of field range that was nearest lens 508. Using a software tool, the pathologist may then be permitted to “peel back” that first image to reveal a second image that focuses on the next lower depth of field range. The pathologist may continue to “peel back” images until reaching an image that is focused at the depth of field of interest.

Alternatively, multiple images for a single slide may be digitally combined to create a single image that is in focus at every depth of field range. More specifically, the portion of each image that is in focus may be determined by identifying high contrast transitions in the image. The in-focus portion of each of the images may then be combined to create a single image which is in focus at every depth of field range.

Returning to FIG. 4, at 414, the digital images are preferably indexed and stored in database 208 of server computer 202. In step 416, these images are retrieved from database 208 and displayed to a pathologist on a display screen. It should be understood that server computer 202 may be located in the same facility with workstation computer 504 (e.g., at a single pathology lab) or may be maintained at a separate location. In a preferred embodiment, tissue slides 506 are imaged at a regional pathology lab and compressed and loaded onto database 208. Alternatively, slides 506 may also be sent to a central facility for imaging (e.g., the location of the server computer 202).

As noted above, in a preferred embodiment, the image retrieved from database 208 is digitally magnified before display to the pathologist. This provides significant benefits over the optical microscopy methods of the prior art including increased field of view and depth of field. Thus, for example, the present system allows a diagnosing pathologist to view an entire tissue sample on a display 228 (shown in FIG. 2) at one time, without having to move a tissue slide under a microscope's limited field of view.

After the pathologist has completed and stored the diagnostic report, the report may be reviewed by tumor boards, specialists, and other authorized users by accessing the digital image and associated report stored in database 208.

While the present invention has been described with reference to the preferred embodiments, those skilled in the art will recognize that numerous variations and modifications may be made without departing from the scope of the present invention. Accordingly, it should be clearly understood that the embodiments of the invention described above are not intended as limitations on the scope of the invention, which is defined only by the following claims.

What is claimed is:

1. A method for providing centralized anatomic pathology services comprising:
providing a plurality of regional pathology laboratories, each laboratory servicing a geographic region;
maintaining in memory pathology information accessible by pathologists associated with any of the plurality of regional laboratories via a communications link;
collecting tissue requiring pathology processing from a medical entity located in a first geographic region, the step of collecting being performed by an agent of the regional pathology laboratory located in the first geographic region;
processing the tissue to create a tissue slide;
creating a digital, diagnostic quality image of the tissue slide;
storing the image in the memory; and
providing access to the stored diagnostic image to a pathologist via the communications link, to enable diagnosis by the pathologist without physical possession of the slide.

2. The method of claim 1, further comprising compressing the digital, diagnostic quality image prior to storing the image in the memory.

3. The method of claim 2, further comprising using a wavelet based compression algorithm to compress the image.

4. The method of claim 1, wherein the step of creating a digital image of the tissue slide includes exposing the tissue slide to non-visible electro-magnetic radiation.

5. The method of claim 1, further comprising storing analysis and annotations prepared by the pathologist relating to the diagnostic image in the memory.

6. The method of claim 5, wherein the analysis and annotations includes auditory comments.

7. The method of claim 5, wherein the analysis and annotations includes an annotated overlay for the digital image.

8. The method of claim 1, wherein the pathologist is remotely located with respect to the first geographic region.

9. The method of claim 1, wherein the diagnosis is a primary diagnosis.

10. The method of claim 1, wherein the diagnosis is a secondary diagnosis.

11. The method of claim 10, further comprising providing access to prior analysis and annotations, stored in the memory, relating to the diagnostic image to the pathologist to enable the secondary diagnosis by the pathologist.

12. The method of claim 1, wherein the memory storing pathology information includes diagnostic treatment information.

13. The method of claim 12, further comprising providing diagnostic treatment information relating to the diagnosis to an authorized user.

14. The method of claim 1, wherein the geographic region serviced by the regional laboratory includes areas within a radius of approximately one-hundred miles from the regional laboratory.

15. The method of claim 1, wherein the step of creating a digital, diagnostic quality image of the tissue slide includes combining optical magnification and non-empty digital magnification.

16. The method of claim 1, wherein the step creating a digital, diagnostic quality image of the tissue slide includes capturing multiple images of the tissue slide, each of which is in focus for a particular depth of field range.

17. The method of claim 16, wherein the step of creating a digital, diagnostic quality image of the tissue slide further includes digitally combining the multiple images of the tissue slide to create a single image that is in focus at every depth of field range.

18. The method of claim 1, further comprising providing simultaneous access to the diagnostic quality, digital image to two or more pathologists via the communications link to enable simultaneous diagnosis by the pathologists without physical possession of the slide.

19. A system for providing centralized anatomic pathology services comprising:
a scanning device for creating a digital, diagnostic quality image of a tissue slide;
a database configured to store pathology information, including the digital, diagnostic quality image;
a processor configured to compress the digital, diagnostic quality image prior to storage in the database and provide access to pathology information, including the image, to an authorized user to enable diagnosis by the user without physical possession of the slide; and
a communications link connecting a plurality of regional pathology laboratories and other authorized users to the server computer, where each laboratory services a defined geographic region.

20. The system of claim 19, wherein the pathology information stored in the database further includes diagnostic treatment information.

21. The system of claim 19, wherein the processor is further configured to provide diagnostic treatment information relating to the diagnosis to an authorized user.

22. The system of claim 19, wherein the database is further configured to store analysis and annotations relating to the image.

23. The method of claim 22, wherein the analysis and annotations includes auditory comments.

24. The method of claim 22, wherein the analysis and annotations includes an annotated overlay for the digital image.

25. The system of claim 19, wherein the scanning device further comprises
a charge coupled device (CCD);
a biaxial positioning device;
a lens;
a light source;
a collimator for collimating the light source toward the tissue slide and CCD; and
a controller for configuring the CCD, the positioning device, the light source, and the collimator.

26. The system of claim 25, wherein the CCD is a linear CCD.

27. The system of claim 25, wherein the lens has a higher resolution than the human eye can resolve.

28. The system of claim 25, wherein the biaxial positioning device moves the CCD with respect to the tissue slide.

29. The system of claim 25, wherein the biaxial positioning device moves the tissue slide with respect to the CCD.

30. A method for providing centralized anatomic pathology services to a plurality of health institutions, each health institution being located in a geographic region, comprising:
providing a network of regional pathology laboratories, each laboratory servicing a geographic region;
collecting tissue requiring pathology processing from each of a plurality of health institutions located in a first geographic region, the step of collecting being performed by an agent of the regional pathology laboratory located in the first geographic region;
transporting the collected tissue to the regional pathology laboratory located in the first geographic region;
processing the tissue to create a tissue slide; and
analyzing the tissue slide to render a diagnosis, the step of analyzing being performed by a pathologist; and
maintaining a centralized records database of diagnostic information accessible by authorized users via a communications link.

31. The method of claim 30, further comprising storing analysis and annotations prepared by the pathologist relating to the tissue slide in the centralized records database.

32. The method of claim 30, wherein the step of analyzing the tissue slide includes the step of viewing a digital, diagnostic quality image of the tissue slide.

33. The method of claim 30, wherein the pathologist is remotely located with respect to the first geographic region.

34. The method of claim 30, wherein the geographic region serviced by the regional laboratory includes areas within a radius of approximately one-hundred miles from the regional laboratory.

35. The method of claim 30, further comprising collecting diagnostic treatment information and providing diagnostic treatment information to inquiring patients and physicians.

36. The method of claim 30, wherein the step of creating a digital, diagnostic quality image of the tissue slide includes combining optical magnification and non-empty digital magnification.

37. A method for providing centralized anatomic pathology services to a plurality of health institutions, each health institution being located in a geographic region, comprising:
providing a plurality of regional pathology laboratories, each laboratory servicing a geographic region;
establishing a relationship with a health institution to provide the health institution with anatomic pathology services without acquiring an existing pathology practice;
collecting tissue requiring pathology processing from each of a plurality of health institutions located in a first geographic region, the step of collecting being performed by an agent of the regional pathology laboratory located in the first geographic region;
transporting the collected tissue to the regional pathology laboratory located in the first geographic region;
processing the tissue to create a tissue slide; and
analyzing the tissue slide to render a diagnosis, the step of analyzing being performed by a pathologist.

38. The method of claim 37, further comprising maintaining a centralized records database accessible by all regional pathology laboratories in the network.

39. A system for creating diagnostic quality, digital pathology images comprising:
a charge coupled device (CCD);
a biaxial positioning device;
a lens having a higher resolution than the human eye can resolve;
a light source;
a collimator for collimating the light source toward the tissue slide and CCD; and
a controller for configuring the CCD, the positioning device, the light source, and the collimator.

40. The system of claim 39, wherein the CCD is a linear CCD.

41. The system of claim 39, wherein the light source provides non-visible electromagnetic radiation.

42. The system of claim 39, wherein the light source provides visible electromagnetic radiation.
